(19) United States
(12) Patent Application Publication
(10) Pub. No.: US 2008/0014231 A1
(43) Pub. Date: Jan. 17, 2008

(54) Treatment articles capable of delivering intensive care and overall treatment simultaneously

(75) Inventor: Toshihiko Okano, Kobe (JP)

Correspondence Address: THE PROCTER & GAMBLE COMPANY;INTELLECTUAL PROPERTY DIVISION - WEST BLDG., WINTON HILL BUSINESS CENTER - BOX 412, 6250 CENTER HILL AVENUE, CINCINNATI, OH, 45224, US

(73) Assignee: The Procter & Gamble Company

(21) Appl. No.: 11/827,939

(22) Filed: Jul. 13, 2007

Related U.S. Application Data
(60) Provisional application No. 60/831,106, filed on Jul. 14, 2006.

(51) Int. Cl. A61K 8/02; A61K 38/02; A61K 9/70; A61P 17/00; A61Q 19/00

(52) U.S. Cl. 424/401; 424/400; 514/2

(57) ABSTRACT

The present invention relates to treatment articles capable of delivering intensive care and overall care simultaneously. The articles comprise a first substrate, a second substrate which substantially covers the first substrate at-use, a first treatment composition and a second treatment composition wherein the delivery of the skin care active to the skin is initiated by the treatment composition. The articles also comprises a first substrate which contains a first treatment composition, a second substrate which substantially cover the first substrate at-use and a treatment composition wherein delivery of the first treatment composition is initiated by the second treatment composition. Preferred articles of the present invention comprise facial treatment masks.

### CROSS REFERENCE TO RELATED APPLICATION

This application claims the benefit of U.S. Provisional Application No. 60/831,106 filed on Jul. 14, 2006.

### FIELD OF THE INVENTION

The present invention relates to treatment articles which are capable of delivering intensive care and overall care simultaneously.

### BACKGROUND OF THE INVENTION

Cosmetic treatment articles including masks designed for providing treatment to the skin are known in the art. Such articles are typically made of a substrate and a treatment composition soaked in the substrate, wherein the substrate is a porous, water insoluble substrate. The articles are applied to mammalian skin where the treatment composition is transferred. These cosmetic articles are commonly applied to the skin for cleansing and the overall care of the skin as well as to improve the health and physical appearance of the skin by providing intensive care.

In facial treatment, certain areas of the face require intensive care as compared to other areas. For example, areas more vulnerable to wrinkles such as areas surrounding the eyes need more intensive anti-aging care, whereas hyperpigmentated areas caused by such as age spots, freckles and discoloration associated with sunlight, skin aging and environmental damage need more intensive whitening care. To provide intensive care to target areas, smaller size masks for target areas such as eye masks and spot whitening products are provided. Since such intensive care products are applied only to target areas, and other facial area is left without treatment during mask application, a follow-up application with a full facial mask is needed to provide overall skin care benefit to the entire facial area.

Meanwhile, in order to maximize skin benefits, it is recommended to use multiple skin benefit agents together. While masks are suitable for delivering a variety of skin benefit agents to the skin, there are certain skin benefit agents and chemical compounds that are difficult to be formulated together due to instability, colorization, etc. For example, skin benefit agents having different stable pH ranges are difficult to be formulated in the same treatment composition due to a stability problem. Similarly certain compounds react with other compounds and produce unaesthetic color and/or odor. These factors may significantly affect product performance and/or aesthetics, and limit the scope of compounds that can be provided by a single treatment article.

PCT application WO 2004/110414 relates to preparation at-use device comprising pre-formed hydrogel product for effectively delivering skin benefit agents to the skin. While this device can effectively deliver compounds which are unstable when contacting with hydrogel, it only can provide a single skin care benefit to the application skin area, and does not provide customized benefit to target areas and overall treatment for other area simultaneously.

Delivery of skin care actives to the skin via a mask providing wet, typically aqueous environment, is advantageous in that the skin is exposed to an abundant amount of such agents over a lengthy period of time. Since the usage encourages the consumer to sit or lie down for a certain period of time without significant moving to secure sufficient contact between the mask and the skin, multiple applications to provide various skin care benefits to the skin bring consumers' inconvenience.

Accordingly, the need remains for a treatment article which simultaneously delivers intensive care to target areas and overall care to other areas thereby delivering consumers' convenience due to saving their time and efforts in mask applications. None of the existing art provides all the benefits and advantages of the present invention.

### SUMMARY OF THE INVENTION

The present invention relates to treatment articles comprising a first substrate, a second substrate and treatment compositions. Treatment articles according to the present invention simultaneously deliver intensive care and overall care in a single application.

The present invention is directed to a treatment article comprising a first substrate; a second substrate which has a shape to substantially cover the first substrate at-use; a first treatment composition; and a second treatment composition, wherein the second substrate is a water-insoluble substrate.

The present invention is also directed to a treatment article comprising a first substrate containing a first treatment composition; a second substrate which has a shape to substantially cover the first substrate at-use; and a second treatment composition, wherein the second substrate is a water-insoluble substrate and delivery of the first treatment composition to the skin is initiated by contacting the first substrate with the second treatment composition.

The present invention is further directed to a cosmetic treatment method comprising the steps of providing a treatment article comprising a first substrate, a second substrate, a first treatment composition and a second treatment composition, wherein the second substrate is a water-insoluble substrate; applying the first substrate with the first treatment composition to a target area of the skin; applying the second substrate with the second treatment composition to the skin in a way to cover the first substrate and other areas of the skin; and allowing the treatment article to remain on the skin for a period of time effective to provide cosmetic treatment.

The present invention is further directed to a cosmetic treatment method comprising the steps of providing a treatment article comprising a first substrate, a second substrate, a first treatment composition and a second treatment composition, wherein the second substrate is a water-insoluble substrate; applying the first substrate with the first treatment composition on to a part of the second substrate which is to be located on a target area of the skin; applying the second substrate with the second treatment composition to the skin in a way to locate the first substrate on to the target area of the skin and the other part of the second substrate covers other areas of the skin; and allowing the treatment article to remain on the skin for a period of time effective to provide cosmetic treatment.

These and other features, aspects, and advantages of the present invention will become better understood from a reading of the following description, and appended claims.

### BRIEF DESCRIPTION OF THE DRAWINGS

While the specification concludes with claims particularly pointing out and distinctly claiming the invention, it is believed that the present invention will be better understood from the following description taken in conjunction with the accompanying drawings.

FIG. 1 is a planar view of a preferred embodiment of the second substrate of the present invention; and

FIG. 2 is a planar view of a preferred embodiment of the treatment article of the present invention.

### DETAILED DESCRIPTION OF THE INVENTION

According to the present invention, the articles simultaneously provide intensive care to a target area of the skin and overall care other areas of the skin during a single application.

While the article of the present invention will be described in the context of a facial mask, one of ordinary skill in the art will recognize the ability to and be able to readily adapt the present invention to treatment articles in general. As used herein, the term “treatment article” and “cosmetic treatment article” refers to devices which are adapted for application to the body, and in particular, to the human body.

In addition, “comprising” means that other steps and other ingredients which do not affect the end result can be added. This term encompasses the terms “consisting of” and “consisting essentially of”.

As used herein, “topical application” means to apply or spread a material onto the surface of the skin.

As used herein, “cosmetically acceptable” means that the compositions or components thereof so described are suitable for use in contact with human skin without undue toxicity, incompatibility, instability, allergic response, and the like.

As used herein, “mixtures” is meant to include a simple combination of materials and any compounds that may result from their combination.

As used herein, “intensive care” means accumulated skin care benefits delivered by the combination of the first treatment composition and the second treatment composition of the present invention.

As used herein, “overall care” means skin care benefits delivered by the second treatment composition of the present invention.

As used herein, “other areas” means skin areas where the first treatment composition of the present invention is not applied.

All ingredients such as actives and other ingredients useful herein may be categorized or described by their cosmetic and/or therapeutic benefit or their postulated mode of action. However, it is to be understood that the actives and other ingredients useful herein can, in some instances, provide more than one cosmetic and/or therapeutic benefit or operate via more than one mode of action. Therefore, classifications herein are made for the sake of convenience and are not intended to limit an ingredient to the particularly stated application or applications listed.

All percentages, parts and ratios are based upon the total weight of the compositions of the present invention, unless otherwise specified. All such weights as they pertain to listed ingredients are based on the active level and, therefore do not include carriers or by-products that may be included in commercially available materials.

The articles of the present invention preferably comprise one or more first substrates, one or more second substrates and one or more treatment compositions. Herein, “a substrate” or “the substrate” refers to the first substrate and/or the second substrate.

### First Substrate

In the present invention, the first substrate can be selected from water-insoluble substrates and water-soluble films.

Water-Soluble Film

By “water-soluble”, it is meant that the water-soluble film can dissolve in or readily break apart upon immersion in water. Ideally, the water-soluble film should not be present on the skin for long periods when water or a treatment composition is applied to the water-soluble film. A wide variety of materials can be used as the water-soluble film. The following nonlimiting characteristics are desirable: (i) appropriate dissolution time, (ii) sufficient flexibility and (iii) sufficient strength to handle.

The water-soluble films may be comprised of a variety of materials both natural and synthetic. Nonlimiting examples of suitable water-soluble polymers include alkylcelluloses such as methylcellulose; hydroxyalkylcelluloses such as hydroxybutyl cellulose, hydroxyethylmethylcellulose, hydroxyethylcellulose, hydroxypropylcelluose; hydroxyalkylalkylcelluloses such as hydroxypropylmethylcellulose; carboxyalkylcelluloses such as carboxymethylcellulose; alkali metal salts of carboxyalkylcelluloses such as sodium carboxymethylcellulose; carboxyalkylalkylcelluloses such as carboxymethylethyl cellulose; carboxyalkylcellulose esters; starches; gelatins; agars; sccharides; pectins such as sodium carboxylmethyl amylopectin; seaweed extracts; chitin derivatives such as chitosan; cationic polymers such as polyquaternium-10, polyquaternium-16, polyquaternium-28, polyquaternium-44, polyquaternium-46, polyquaternium-55, vinylpyrrolidone/dimethylaminopropyl methacrylamide copolymer, vinylpyrrolidone/dimethylaminoethyl methacrylate copolymer, polysaccharides such as alginic acid, alkali metal and ammonium salts thereof, carrageenans, galactomannans, traganth, agar, gum Arabicum, guar gum and xanthan gum, polyacrylic acids and salts thereof, polymethacrylic acids and salts thereof, including methacrylate-vinyl alcohol copolymers, polyvinyl alcohol, polyvinyl alcohol copolymers or derivatives, vinyl acetate-vinyl alcohol polymers, polyvinylpyrrolidone, hydrolyzed polyvinylpyrrolidone, polyacrylamide, poly(methacrylamide), dextran, polyethylene glycol, and polyoxyethylene and polyoxypropylene block copolymers and mixtures thereof.

Water-soluble films may be made by using various conventional water-soluble film making technologies known in the art such as disclosed in U.S. Pat. Nos. 6,596,298 and 6,419,903, and Japanese Patent Application Laid-Open No. 2005-112957. For example, film components may be dispersed in water or other solvent and dried into film form. In an alternative, dry components may be blended and then dispersed with any additional film components in water or other solvent and dried into film form. Films may be formed from such dispersions or solutions by shaping it into a solidified form of a suitable thickness by any techniques known in the art including, but not limited to, wet casting, freeze-drying and extrusion molding.

The water-soluble film of the present invention can consist of a single layer or multiple layers. When the water-soluble film consists of multiple layers, films can be coated or spayed with another water-soluble film dispersion or solution.

Water-soluble films useful in the present invention can also be obtained from a variety of commercial sources. Nonlimiting examples of suitable water-soluble films useful herein include films used for Listerin Pocketpack (Pfizer, U.S.A.), Breath Care Film (Kobayashi Seiyaku, Japan), Metolose (ShinEtsu Chemical, Japan), Porphyran film (Shirako, Japan) and oblate.

Preferably the water-soluble film may completely dissolve or readily break apart by contact with water within 0.1-60 minutes, within preferably 3-30 minutes, more preferably within 5-15 minutes. If the film dissolves too fast, it may be inconvenient in consumers' use since it is unstable against the moisture from air or difficult to handle. If it dissolves too slowly, application time would lengthen. The water-soluble film should be flexible enough to fit along the surface of the skin on which it is applied. Yet, minimum strength is required so that the film does not rupture during handling, for example, when the consumer handles the film. Preferably the water-soluble film may have thickness in the range of 1-300 μm, preferably in the range of 10-100 μm, more preferably in the range of 30-60 μm.

Water-Insoluble Substrate

By “water-insoluble”, it is meant that the substrate does not dissolve in or readily break apart upon immersion in water. The water-insoluble substrate is the implement or vehicle for delivering the treatment composition to the skin.

A wide variety of materials can be used as the water-insoluble substrate. The following nonlimiting characteristics are desirable: (i) sufficient wet strength for use, (ii) sufficient softness, (iii) sufficient thickness, (iv) appropriate size, (v) air permeability, (vi) hydrophilicity, and (vii) its ability to take permanent incremental strain, that is, its ability to be incrementally stretched.

Nonlimiting examples of suitable water-insoluble substrates which meet the above criteria include nonwoven substrates, woven substrates, hydroentangled substrates, air entangled substrates, natural sponges, synthetic sponges, polymeric netted meshes, and the like. Preferred embodiments employ nonwoven substrates since they are economical and readily available in a variety of materials. By “nonwoven”, it is meant that the layer is comprised of fibers which are not woven into a fabric but rather are formed into a sheet, mat, or pad layer.

The water-insoluble substrates may be comprised of a variety of materials both natural and synthetic. Nonlimiting examples of natural materials useful in the present invention include: silk fibers; keratin fibers such as wool fibers and camel hair fibers; and cellulose fibers such as wood pulp fibers, cotton fibers, hemp fibers, jute fibers, and flax fibers. Nonlimiting examples of synthetic materials useful in the present invention include: acetate fibers; acrylic fibers; cellulose ester fibers; polyamide fibers; polyester fibers such as polyethylene terephthalate fibers; polyolefin fibers such as polypropylene fibers and polyethylene fibers; polyvinyl alcohol fibers; rayon fibers; and polyurethane foam.

Water-insoluble substrates useful in the present invention can also be obtained from a wide variety of commercial sources. Nonlimiting examples of suitable nonwoven substrates useful herein include the following. Havix 2360 available from Havix, Japan. Further suitable is TT463Q60 available from Kuraray, Japan. Also suitable are WALKISOFT®, a cellulose substrate available from Walkisoft U.S.A.; NOVONET® 149-801 and 149-191 available from Veratec, Inc. Walpole, Mass.; KEYBAK® 951V and 1368, available from PGI/Chicopee, Dayton, N.J.; RMT-90 RFP-90 and DFS(SH)T-70 available from Daiwabo K.K. of Japan. Further suitable substrates include Kuraray TT463Q60.

The water-insoluble substrate is flexible enough such that, when impregnated with a treatment composition, it readily fits along the skin, yet is strong enough so that it does not easily tear or crumble upon use. Preferably, the substrate has a thickness of from about 100 μm to about 1 cm, more preferably from about 300 μm to about 3 mm, depending on the material for making the substrate, and use and characteristic of the product.

The water-insoluble substrate can be made solely of hydrophilic material, or made of a mixture of hydrophilic material and hydrophobic material. The water-insoluble substrate of the present invention can consist of a single layer or multiple layers. In one preferred embodiment, the substrate is made of at least partially by hydrophilic materials selected from cotton, pulp, rayon, and mixtures thereof. By partially, it is meant to encompass following situations: where one layer of a hydrophilic material is used for a single layered substrate; where at least one layer of a hydrophilic material is used for a multiple layered substrate; where one layer of a mixture of the hydrophilic material and another material is used for a single layered substrate; and where at least one layer of a mixture of the hydrophilic material and another material is used for a multiple layered substrate.

When the water-insoluble substrate consists of multiple layers, the substrates can include films and other nonfibrous materials. In one embodiment, the substrate may also be laminated with polymeric film on the substrate, coating the substrate, or heat sealing the substrate. The resulting substrate with the laminated polymeric film, coating or heat sealing comprises an occluded side on one side of the substrate, which faces away from the skin, and a skin facing side that is positioned on the skin surface. By having a substrate with an occluded side, the substrate acquires low air permeability. By “low air permeability” it is meant that the side of the substrate having the film, coating or heat sealing allows very little air to enter into the substrate and very little vapor to escape from the substrate. Preferably the air permeability is less than about 5 mg/cm²/min, more preferably between about 0.01 mg/cm²/min and about 4.8 mg/cm²/min. The air permeability can be measured by taking the weight of a fully saturated sample of the substrate and weighing the substrate after it is exposed to the atmosphere.

### Second Substrate

In the present invention, the second substrate is a water-insoluble substrate. Descriptions and examples of water-insoluble substrates disclosed above under the title of Water-Insoluble Substrate are also applied for the second substrate.

### Treatment Composition

The articles of the present invention include one or more treatment compositions for application to the skin. Herein, “a treatment composition” or “the treatment composition” refers to both the first treatment composition and a second treatment composition or either of them.

The treatment composition may be provided on the substrate via a variety of means. The treatment composition may be provided in liquid form and soaked into the water-insoluble substrate. The water-insoluble substrate may then be packaged and sealed for delivery to consumers and immediate use upon opening of the sealed package. Alternatively, the treatment composition may be dried following application for re-wetting by the consumer prior to application. In yet another embodiment of the present invention, the treatment composition and the water-insoluble substrate may be packaged separately to be combined by the consumer prior to application to the skin. In such instances the treatment composition may be provided either dry or in liquid form. The treatment composition may be provided to the water-soluble film by impregnating the treatment composition into the film, for example, by admixing the treatment composition with the film forming dispersions or solutions and then forming the film, or by exposure of the film to the treatment composition under conditions which cause a skin benefit agent to be impregnated into the film. The treatment composition may be provided to the water-soluble film by depositing the treatment composition upon the surfaces of films as known, for example printing, e.g. silo screen printing, passing between impregnated rollers, dosing, a pump and nozzle, spraying, dipping etc. When a water-soluble film is a multilayer type film, at least one layer may contain a different treatment composition from other layer.

The treatment composition of the present invention preferably includes a skin benefit agent to provide a benefit to the skin, in an amount from about 0.01 wt % to about 30 wt %, preferably from about 0.1 wt %˜about 20 wt % by weight of the composition. The skin benefit agent of the present invention includes those as known in the art and includes anti-oxidants, cleansing agents, free radical scavengers, moisturizers, chronic whitening agents, anti-acne agents, anti-dandruff agents, anti-aging agents, softeners, anti-wrinkle agents, keratolic agents, anti-inflamatory agents, oily components, skin texture treatment agents, flavanoids, fresheners, healing agents, liporegulators, vascular protectors, anti-bacterial agents, anti-fungal agents, anti-perspirant agents, deodorants, skin conditioners, anesthetics, nourishing agents, sebum absorbers, and moisture absorbers. The ingredients are described in more detail herein.

Anti-oxidants and radical scavengers are especially useful for providing protection against UV radiation which can cause increased scaling or texture changes in the stratum corneum and against other environmental agents which can cause skin damage and may be optionally included in the treatment composition. Anti-oxidants and radical scavengers such as tocopherol (vitamin E), tocopherol sorbate, tocopherol acetate, other esters of tocopherol, butylated hydroxy benzoic acids and their salts, 6-hydroxy-2,5,7,8-tetramethylchroman-2-carboxylic acid (commercially available under the tradename Trolox®), gallic acid and its alkyl esters, especially propyl gallate, uric acid and its salts and alkyl esters, sorbic acid and its salts, amines (i.e., N,N-diethylhydroxylamine, amino-guanidine), sulfhydryl compounds (i.e., glutathione), dihydroxy fumaric acid and its salts, lycine pidolate, arginine pilolate, nordihydroguaiaretic acid, bioflavonoids, lysine, methionine, proline, superoxide dismutase, silymarin, tea extracts, grape skin/seed extracts, melanin, and rosemary extracts may be used. Preferred anti-oxidants/radical scavengers are selected from flavonoids and tocopherol sorbate and other esters of tocopherol, more preferably tocopherol sorbate. For example, the use of tocopherol sorbate in topical compositions and applicable to the present invention is described in U.S. Pat. No. 4,847,071, Bissett et al, issued Jul. 11, 1989.

Chronic whitening agents useful herein refer to active ingredients that not only alter the appearance of the skin, but further improve hyperpigmentation as compared to pre-treatment. By definition, chronic is referred to continued topical application of the composition over an extended period during the subject's lifetime, preferably for a period of at least about one week, more preferably for a period of at least about one month, even more preferably for at least about three months, even more preferably for at least about one year. Typically, applications would be on the order of about once per day over such extended periods, while application rates can vary from about once per week up to about three times per day or more.

Useful chronic whitening agents useful herein include ascorbic acid compounds, vitamin B₃ compounds, azelaic acid, butyl hydroxy anisole, gallic acid and its derivatives, glycyrrhizinic acid, hydroquinoine, kojic acid, arbutin, mulberry extract, ergothioneine, and mixtures thereof. Among them, preferred are ascorbic acid compounds, vitamin B₃ compounds, and mixtures thereof. Use of combinations of chronic whitening agents are believed to be advantageous in that they may provide whitening benefit through different mechanisms.

Anti-acne actives can be effective in treating and preventing acne vulgais, a chronic disorder of the pilosebaceous follicles. Preferred anti-acne agents optionally included in the treatment composition include salicylic acid, 4-methoxysalicylic acid, benzoyl peroxide, lactic acid, metronidazole, panthenol, retinoic acid and its derivaties, sulphur, triclosan, and mixtures thereof.

Anti-aging agents or anti-wrinkle agents useful herein include, for example, peptides. As used herein, “peptides” refers to both the naturally occurring peptides and synthesized peptides, including but not limited to, di-, tri-, tetra-, and pentapeptides and derivatives thereof. Also useful herein are naturally occurring and commercially available compositions that contain peptides. Suitable dipeptides for use herein include Carnosine® (beta-ala-his). Suitable tripeptides for use herein include, gly-his-lys, arg-lys-arg, his-gly-gly. Preferred tripeptides and derivatives thereof include palmitoyl-gly-his-lys, which may be purchased as Biopeptide CL® (100 ppm of palmitoyl-gly-his-lys commercially available from Sederma, France); Peptide CK (arg-lys-arg); PEPTIDE CK+ (ac-arg-lys-arg-NH₂); and a copper derivative of his-gly-gly sold commercially as IAMIN, from Sigma (St. Louis, Mo.). Tetrapeptides and pentapeptides are also suitable for use herein. A preferred commercially available pentapeptide derivative composition is palmitoyl-lys-thr-thr-lys-ser (commercially available from Sederma France). When included in the present compositions, peptides are preferably included in amounts of from about 1×10⁻⁶% to about 10%, more preferably from about 1×10⁻⁶% to about 0.1%, even more preferably from about 1×10⁻⁵% to about 0.01%, by weight of the composition.

The treatment composition of the present invention may contain an anti-inflammatory agent as a skin benefit agent. Anti-inflammatory agents enhance the skin appearance benefits, by for example, contribution of uniformity and acceptable skin tone and/or color and are optionally included in the treatment composition of the present invention as well. Preferably, the anti-inflammatory agent includes a steroidal anti-inflammatory agent and a non-steroidal anti-inflammatory agent. Preferred steroidal anti-inflammatory for use is hydrocortisone. The variety of compounds encompassed by this group is well-known to those skilled in the art. For detailed disclosure of the chemical structure, synthesis, side effects, etc. of non-steroidal anti-inflammatory agents, reference may be had to standard texts, including Anti-inflammatory and Anti-Rheumatic Drugs, K. D. Rainsford, Vol. I-III, CRC Press, Boca Raton, (1985), and Anti-inflammatory Agents, Chemistry and Pharmacology, 1, R. A. Scherrer, et al., Academic Press, New York (1974), each incorporated herein by reference. So-called “natural” anti-inflammatory agents are also useful. Such agents may suitably be obtained as an extract by suitable physical and/or chemical isolation from natural sources (i.e., plants, fungi, by-products of microorganisms).

Oily components useful herein can deliver skin conditioning benefits such as smoothness and softness to the skin. Oily components useful herein include, for example, fatty alcohols, silicone oils, mineral oil, petrolatum, C₇₋₄₀ straight and branched hydrocarbons such as isohexadecane, C₁₋₃₀ alcohol esters such as isopropyl isostearate, glycerides, alkylene glycol esters, propoxylated and ethoxylated derivatives, sugar ester such as sucrose polycottonseedate, vegetable oils such as coconut oil, hydrogenated vegetable oils, animal fats and oils, and C₄₋₂₀ alkyl ethers of polypropylene glycols, C₁₋₂₀ carboxylic acid esters of polypropylene glycols, and di-C₈₋₃₀ alkyl ethers. Hydrophobic nonionic surfactants, which are those being water-insoluble and having an HLB value of less than 10, can be used as oily components. Hydrophobic nonionic surfactants useful herein include, for example, cetearyl glucoside, steareth-2, laureth-4, sucrose cocate, sorbitan monoisostearate, sorbitan diisostearate, sorbitan sesquiisostearate, sorbitan monooleate, sorbitan dioleate, sorbitan sesquioleate, glyceryl monoisostearate, glyceryl diiostearate, glyceryl sesquiisostearate, glyceryl monooleate, glyceryl dioleate, glyceryl sesquioleate, diglyceryl diisostearate, diglyceryl dioleate, diglycerin monoisostearyl ether, diglycerin diisostearyl ether, and mixtures thereof.

The treatment composition may optionally include a skin texture treatment agent. Skin texture treatment agents help repair and replenish the natural moisture barrier function of the epidermis, thereby providing skin benefits such as texture improvement. Skin texture improvement agents useful herein are niacinamide, nicotinic acid and its esters, nicotinyl alcohol, panthenol, panthenyl ethyl ether, n-acetyl cysteine, n-acetyl-L-serine, phosphodiesterase inhibitors, trimethyl glycine, tocopheryl nicotinate, and vitamin B₃ and analogues or derivatives, and mixtures thereof.

Flavonoid compounds useful herein include unsubstituted flavanones, substituted flavanones, unsubstituted flavones, substituted flavones, unsubstituted chalcones, substituted chalcones, unsubstituted isoflavones, and substituted isoflavones. By the term “substituted” as used herein means flavonoid compounds wherein one or more hydrogen atoms of the skeleton structure as described above has been independently replaced with hydroxyl, C₁-C₈ alkyl, C₁-C₄ alkoxyl, O-glycoside, and the like or a mixture of these substituents. Flavonoid compounds particularly useful herein are selected from the group consisting of substituted flavanones, substituted flavones, substituted chalcones, substituted isoflavones, and mixtures thereof. In one particularly preferred embodiment of the present invention, the glycoside flavonoid is selected from the group consisting of glucosyl hesperidin, glucosyl rutin, glucosyl myricitrin, glucosyl isoquercitrin, glucosyl quercitrin, methyl hesperidin, and mixtures thereof. These glucoside flavonoid compounds can be obtained by bio-chemical methods from related natural flavonoid compounds.

The treatment composition of the present invention preferably contains a water-soluble humectant as a skin benefit agent. Water-soluble humectants are preferably included to provide moisturizing benefit to the skin. Further, water-soluble humectants may help the dispersion of the water-soluble thickening agents, and dissolving/dispersion of other components which are relatively difficult to process in an aqueous carrier.

Water-soluble humectants useful herein include polyhydric alcohols such as glycerin, diglycerin, propylene glycol, dipropylene glycol, butylene glycol, hexylene glycol, sorbitol, ethoxylated glucose, 1,2-hexane diol, hexanetriol, erythritol, trehalose, xylitol, maltitol, maltose, glucose, fructose, sodium chondroitin sulfate, sodium hyaluronate, sodium adenosin phosphate, sodium lactate, pyrrolidone carbonate, glucosamine, cyclodextrin, and mixtures thereof. Water-soluble humectants useful herein also include water-soluble alkoxylated nonionic polymers such as polyethylene glycols and polypropylene glycols having a molecular weight of up to about 1000 such as those with CTFA names PEG-200, PEG-400, PEG-600, PEG-1000, and mixtures thereof.

The treatment composition of the present invention may include a skin tone changing agent. The skin tone changing agent may be included in the composition at a level by weight of, from about 0.001% to about 50%, preferably from about 0.1% to about 25%, more preferably from about 1% to about 10%.

The skin tone changing agent useful herein is selected from the group consisting of skin tone changing pigments, reflective particulate material, and mixtures thereof. Skin tone changing agents useful herein are those altering the appearance of the color and/or tone of the skin including, but not limited to, skin whitening. The skin tone changing agents have a particle size of, preferably at least about 100 nm. The skin tone changing pigments useful herein include, for example, talc, mica, silica, magnesium silicate, titanium oxide, zinc oxide, and titanium oxide coated mica.

The treatment compositions of the present invention are preferably in liquid form and include a water-soluble thickening polymer. The water-soluble thickening polymers herein are water-soluble or water miscible polymers, have the ability to increase the viscosity of the composition, and are compatible with other components used in the composition. The water-soluble thickening polymer is selected so that the liquid composition of the present composition has the desired viscosity of from about 500 mPa·s to about 60,000 mPa·s, preferably from about 1000 mPa·s to about 30,000 mPa·s, more preferably from about 2000 mPa·s to about 15,000 mPa·s. A viscosity may be measured by a commercially available viscometer like BROOKFIELD DV II + Viscometer with Helipath T-C bar type spindle (BROOKFIELD ENGINEERING LABORATORIES, INC.) at 5 rpm/min at 25° C. Water-soluble thickening polymers useful herein include anionic polymers and nonionic polymers. The water-soluble thickening polymers useful herein include, for example, acrylic polymers, polyalkylene glycol polymers having a molecular weight of more than about 10000, celluloses and derivatives there of such as hydroxyethyl cellulose, polyvinylpyrrolidone, polyvinyl alcohol, gums such as guar gum and xanthan gum, carragenan, pectin, agar, quince seed (Cydonia oblonga Mill), starch (rice, corn, potato, wheat), algae colloids (algae extract), dextran, succinoglucan, pulleran, carboxymethyl starch, methylhydroxypropyl starch, sodium alginate, and alginic acid propylene glycol esters. Neutralizing agents may be included to neutralize the anionic thickening agents described hereinabove. Nonlimiting examples of such neutralizing agents include sodium hydroxide, potssium hydroxide, ammonium hydroxide, monethanolamine, diethanolamine, triethanolamine, diisopropanolamine, aminomethylpropanol, tromethamine, tetrahydroxypropyl ethylenediamine, and mixtures thereof.

The treatment composition of the present invention comprises optionally a hydrophilic surfactant. The hydrophilic surfactant may be included in the composition at a level by weight of from about 0.01% to about 10%, preferably from about 0.05% to about 5%, more preferably from about 0.1% to about 2%.

Hydrophilic surfactants useful herein are those being water-soluble, and preferably have an HLB value of above 10. Hydrophilic surfactants useful herein include, for example, any cosmetically acceptable surfactants, i.e., nonionic surfactants, cationic surfactants, anionic surfactants, zwitterionic surfactants, amphoteric surfactants, and mixtures thereof. Among them, preferred herein are cosmetically acceptable nonionic surfactants in view of reduced skin irritation and conditioning benefits. Hydrophilic nonionic surfactants useful herein include, for example, PEG-100 stearate, polysorbate-20, polysorbate-60, seteareth-21, isoceteth-20, and oleth-20, laureth-23, ceteareth-12, steareth-100, PEG 40 hydrogenated castor oil, PEG-60 hydrogenated castor oil, and mixtures thereof.

The compositions of the present invention may include a cooling agent. Cooling agent useful herein include natural cooling agents such as menthol, peppermint oil, camphor, borneol, eucalyptol, eucalyptus oil, tea tree oil, ketals, carboxamides, cyclohexanol derivatives, cyclohexyl derivatives and mixtures thereof.

The treatment compositions of the present invention when in their preferred liquid form further include an aqueous carrier. The level and species of the carrier are selected according to the compatibility with other components, and other desired characteristic of the product.

The aqueous carrier may be included in the composition at a level by weight of from about 0.1% to about 30%, preferably from about 1% to about 25%, more preferably from about 30% to about 10%.

Carriers useful in the present invention include water and water solutions of lower alkyl alcohols. Lower alkyl alcohols useful herein are monohydric alcohols having 1 to 6 carbons, more preferably ethanol. Preferably, the aqueous carrier is substantially water. Deionized water is preferably used. Water from natural sources including mineral cations can also be used, depending on the desired characteristic of the product.

The pH of the liquid compositions is preferably from about 4 to about 8. When skin benefit agents are included in the liquid composition, the pH may be adjusted to that which provides optimum efficacy of the skin benefit agents. Buffers and other pH adjusting agents can be included to achieve the desirable pH. Suitable pH adjusters herein include acetates, phosphates, citrates, triethanolamines and carbonates.

In addition to the above described components, the composition of the present invention may further include preservatives and preservative enhancers such as water-soluble or dispersible preservatives.

### Products for Topical Use

In a preferred embodiment, the article of the present invention is a cosmetic treatment article useful for delivering skin benefit agents to the skin.

The article of the present invention is particularly advantageous in delivering skin benefit agents and in preferred embodiments the skin care agent to the skin as the skin is exposed to an abundant amount of such agents over a lengthy period of time. Further, when a second substrate having low air permeability is used, more effective penetration of the skin benefit agents into the skin is expected. The articles of the present invention may also provide emotional benefits to the consumer upon use, such as refreshing feel, and relaxation feel.

In a preferred embodiment, the cosmetic treatment article is a mask comprising a first substrate, a second substrate, a first treatment composition and a second treatment composition wherein the second substrate is a water-insoluble substrate.

In this embodiment, the treatment composition can be provided with the substrate, or be separately provided to be applied to the skin or to the substrate.

In this embodiment, the substrate, either the first substrate or the second substrate, can be made into a wide variety of shapes and forms such as flat pads, thick pads, thin sheets, and sheets of irregular thickness, depending on the desired use and characteristic of the article. The substrate is typically designed to fit the area of the skin to which topical application is desired.

The first substrate can be, for example, square, circular, semicircular, rectangular, oval, rings, crescents, teardrops or other more complex shapes which may be composites of these, for covering areas such as the eye area, eye lids, the nose, the mouth area, the forehead, the cheek, the chin, the entire contour of the face, neck, arm, other specific places of the body, or combinations thereof.

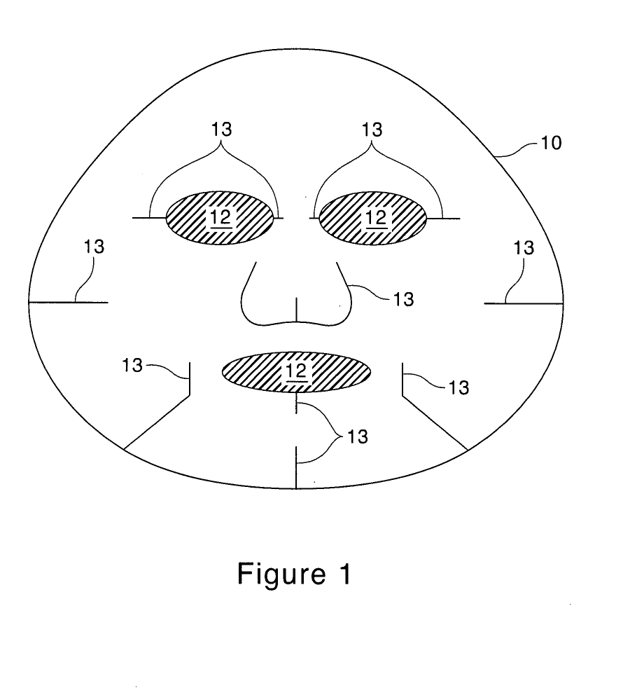

The second substrate can be, for example, the same shape as the first substrate to substantially cover it at-use or a single-piece whole facial mask. However, it will be readily apparent to one of ordinary skill that the articles of the present invention may comprise a single piece or a multi-part article. Referring to FIG. 1, a plane view of a preferred embodiment of a second substrate suitable for a single-piece whole facial mask (10) is depicted. The outer peripheral of the substrate of FIG. 1 is designed to approximately match the contour of the face, with a plurality of openings (12) for the eyes and the mouth, and wherein a plurality of cuttings (13) are made so that the mask fits the nose, cheeks, and the mouth. However, it will be readily apparent to one of ordinary skill that the second substrate of the present invention may comprise a single piece or a multi-part mask. For example, a multi-part mask is configured to cover substantially the whole area of the facial skin, and is made of multiple pieces. Most commonly, multi-part masks comprise two-part masks having a first piece covering the upper area of the face, i.e. the nose and there above, and a second piece covering the lower area of the face, i.e. the upper lip, cheeks and there under. Of course, one of ordinary skill will recognize that articles having three or more pieces may be envisioned. In addition, the exact areas of coverage of multi-part masks as to each individual piece may vary. The second substrate may be so configured to have ears, pulls, or rings for facilitating placement and/or removal of the mask on the skin. The second substrate may have at least one elongation zone wherein at least a portion of the elongation zone has been provided with incremental stretch to provide better contact between the substrate and the skin as disclosed in WO06/053332.

In a preferred embodiment, the first treatment composition of the present invention provides intensive care to a target area of the skin in need of a specific treatment by delivering to the target area additional benefits not delivered by the second treatment composition alone. The first treatment composition may be the same as or differ from the second treatment composition. The first treatment composition may contain a compound which is unstable in contact with the second treatment composition, for example, a compound that enhances syneresis, produces unaesthetic color, taste, flavor, and/or odor, or alters its physical properties or decreases its performance when contacted with the second treatment composition. The first treatment composition may contain a compound which is known to be unstable with water, for example, an easily oxidated water-soluble compound such as ascorbic acid compounds, a heat generating compound such as anhydrous magnesium sulfate, a powder compound, an electrically charged compounds such as an electrolyte and an ionic surfactant, and mixtures thereof.

In another preferred embodiment, the first substrate may be provided as multiple pieces to be applied on target areas in need of a specific treatment. For example, two pieces of the first substrate which may contain a high level of a skin moisture benefit agent are provided to be applied at the cheek locations at use. Similarly, multiple pieces of the first substrate which may contain a high level of a whitening agent are provided to be applied to hyperpigmentated areas at use.

In another preferred embodiment, the first substrate may be provided as multiple pieces to be applied on target areas in need of different specific treatment. In this embodiment, at least a piece of the first substrate may either contain a skin benefit agent which another piece of the first substrate does not contain or contain the same skin benefit agent as another piece of the first substrate contains in a different level. For example, two pieces of a first substrate applied at the cheek locations may contain a high level of a skin moisture benefit agent, while a piece of the first substrate applied to the nose location may contain less skin moisture benefit agent and/or an anti-acne benefit agent. Similarly, a first substrate selectively applied at the eye contour area and/or at the nasolabial fold locations may contain anti-wrinkle agents while multiple pieces of the first substrate applied to hyperpigmentated areas contains a whitening agent. There are many variations for providing a desired benefit agent to a target area of the skin in need of a specific treatment by selectively applying the first substrate with the first treatment composition to the corresponding location of the skin, and all variations and any combination thereof are within the scope of the present invention.

In another preferred embodiment, the first substrate may be a water-insoluble substrate preferably containing the first treatment composition in a liquid form. Preferably the thickness of the first substrate is thinner as compared to the second substrate.

In a highly preferred embodiment, preferably the first substrate is a water-soluble film containing the first treatment composition in a dried form. The water-soluble film dissolves when contacted with the second treatment composition or with water, and delivery of the first treatment composition contained in the water-soluble film to the skin is initiated thereby.

In another embodiment, the article of the present invention may be provided as a product comprising a) a first substrate containing a first treatment composition located in a first compartment and b) a second substrate containing a second treatment composition located in a second compartment wherein the second substrate is water-insoluble and has a shape to substantially cover the first substrate at-use; wherein the first and the second substrates are not contacted prior to use, and the first and second compartments are housed in a single package.

### Method of Use

The article of the present invention is suitable for topical application on human body skin, particularly facial skin.

In another preferred embodiment, a cosmetic article is used to treat the facial skin by the steps of:

a) providing a treatment article comprising a first substrate, a second substrate, a first treatment composition and a second treatment composition wherein the second substrate is a water-insoluble substrate;

b) applying the first substrate with the first treatment composition to a target area of the skin;

c) applying the second substrate with the second treatment composition to the skin to cover the first substrate; and

d) allowing the second substrate to stand on the skin for a period of time effective to provide cosmetic treatment.

In another preferred embodiment, a cosmetic article is used to treat the facial skin by the steps of:

a) providing a treatment article comprising a first substrate, a second substrate, a first treatment composition and a second treatment composition wherein the second substrate is a water-insoluble substrate;

b) applying the first substrate with the first treatment composition to such an area on the second substrate which is located on a target area of the skin;

c) applying the second substrate with the second treatment composition to the skin in a way that the first substrate is located on the target area of the skin; and

d) allowing the second substrate to stand on the skin for a period of time effective to provide cosmetic treatment.

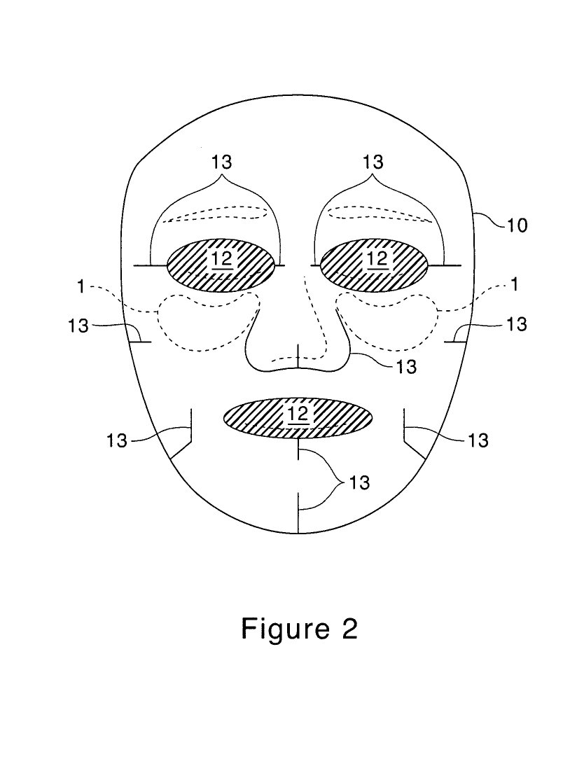

These embodiments to use of the present articles are believed to provide maximum skin care benefit to the consumer, because they can deliver intensive care for a target area of the skin where the first substrate with the first treatment composition is applied and the overall care for the area where the second substrate with the second treatment composition alone is applied. With the first treatment composition the first substrate provides intensive care to the target area. With the second treatment composition, the second substrate provides overall care to the skin. Preferably, the second substrate places to the skin to cover the whole area of the facial skin including the target area where the first substrate is located with areas of the eyes, nostrils and/or mouth opened. The areas of the eyes, nostrils and/or mouth can have a slit instead of opening. Referring to FIG. 2, two pieces of the first substrate (1) with a first treatment composition are applied to areas of eyes and the second substrate (10) with a second treatment composition is applied on top of the first substrate in a way to cover the whole area of the facial skin including the areas below eyes where the first substrate was applied.

In these embodiments, the article is allowed to stand on the skin for a period of time no longer than until any portion of the treatment composition is dried whereupon the article is removed from the skin. In the preferred embodiment of a facial mask, the second substrate mask is soaked or otherwise provided with the second treatment composition and the mask is fitted to the facial skin by gently placing on the skin.

As used herein, “dried” refers to a state wherein water and other volatile components such as perfume, if included, evaporates from the water insoluble substrate mask, thereby leaving the substrate significantly less capable of delivering the treatment composition to the skin. Thus, once a portion of the mask is dried, even distribution of the composition cannot be expected. Further, when dried, the mask provides an unpleasant stiff and tough feeling to the skin when applied.

Because the composition of the present invention is easily dried via exposure to regular atmospheric conditions, articles which are provided wet are preferably housed in a hermetically sealed package during storage.

The period of time required until dried portions appear will depend on the atmosphere in which the use takes place, i.e. temperature, humidity, air circulation; and the structure and body temperature of the consumer. Typically, the composition should be designed so that no dried portions appear within a period of about 15 minutes when used in room temperature at a humidity of about 50%. When an insoluble substrate having low air permeability is used, the period of time by which the composition is dried can be prolonged, preferably from about 5 to about 45 minutes.

### EXAMPLES

The following examples further describe and demonstrate embodiments within the scope of the present invention. The examples are given solely for the purposes of illustration and are not to be construed as limitations of the present invention, as many variations are possible without departing from the spirit and scope of the inventions. Where applicable, ingredients are identified by chemical or CTFA name, or otherwise defined below.

### Examples 1-8 Water-Soluble Films Suitable for the First Substrate

Monolayer water-soluble films may be prepared according to Examples 1-4 and 8. The monolayer water-soluble film of Example 1 may be produced as follow:

### Step 1: Water and glycerin are mixed homogeneously to obtain a mixture.

### Step 2: Sodium alginate and waxy corn starch are added to the mixture of Step 1 to obtain a mixture and it is gelatinized by heating up to 80° C.

### Step 3: The gelatinized mixture of Step 2 is cooled down to 40° C., and is cast onto a PET film, and is dried at 80° C.

The film of Example 2 may be produced by adding ascorbyl glucoside solution which is prepared by dissolving ascorbyl glucoside in water and controlling pH to 5.5 to the gelatinized second mixture which is cooled down to 60° C. Then, Step 3 in Example 1 is conduced. The film of Example 3 may be produced as described in Example 2 with substitution of ascorbyl glucoside with N-acetyl glucosamine. The film of Example 4 may be prepared by mixing all components, casting the obtained mixture of layer B onto a PET film, and drying it at 80° C.

Multilayer water-soluble films may be prepared according to Examples 5-7. The film of Example 5 may be prepared by casting layer B with components as set in Table 1 onto a PET film, drying it at 80° C., casting layer A as described in Example 2 on to the layer B instead of a PET film. The films of Examples 6 and 7 may be produced as described in Example 4.

TABLE 1
Water-Soluble Films

| | Ingredient (weight percentage) | Ex. 1 | Ex. 2 | Ex. 3 | Ex. 4 | Ex. 5 | Ex. 6 | Ex. 7 | Ex. 8 |
|---|---|---|---|---|---|---|---|---|---|
| layer A | Water | 32 | 32 | 32 | — | 32 | 32 | 32 | — |
| | Glycerin | 5 | 5 | 5 | — | 5 | 5 | 5 | — |
| | Sodium Alginate *1 | 5 | 5 | 5 | — | 5 | 5 | 5 | — |
| | Waxy Corn Starch *2 | 58 | 56 | 56 | — | 56 | 56 | 56 | — |
| | Ascorbyl Glucoside *3 | — | 2 | — | — | 2 | — | 2 | — |
| | N-Acetyl Glucosamine *4 | — | — | 2 | — | — | 2 | — | — |
| layer B | High MW Starch *5 | — | — | — | 8 | 8 | 8 | 8 | — |
| | Hydroxypropylmethylcellulose *6 | — | — | — | — | — | — | — | 8 |
| | Glycerin | — | — | — | 6 | 6 | 6 | 6 | 2 |
| | Water | — | — | — | 86 | 86 | 86 | 84 | 88 |
| | N-Acetyl Glucosamine *4 | — | — | — | — | — | — | 2 | 2 |

Definitions of Components
*1 Sodium Alginate: available from KIMICA
*2 Waxy Corn Starch: available from National Starch
*3 Ascorbyl Glucoside: Ascorbic Acid 2-Glucoside available from Hayashibara
*4 N-Acetyl Glucosamine: available from General Topics
*5 High MW Starch: available from National Starch
*6 Hydroxypropylmethylcellulose: available from Shinetsu Chemical Co., Ltd.

### Examples 9-16

The Second substrates containing treatment compositions of Examples 9 through 16 are made of about 2.5 g of substrate RFP-90 available from Daiwabo, cut and shaped according to FIG. 1 and soaked with 30 g each of the liquid compositions specified below. The mask compositions can also be made of 3.5 g of cotton substrate instead of the substrate specified above.

TABLE 2
Treatment Compositions

| Components | Ex. 9 | Ex. 10 | Ex. 11 | Ex. 12 | Ex. 13 | Ex. 14 | Ex. 15 | Ex. 16 |
|---|---|---|---|---|---|---|---|---|
| Titanium dioxide *1 | 0.3 | 0.3 | 0.1 | 0.1 | 0.6 | 0.8 | — | 0.3 |
| Resistant starch *2 | — | — | — | — | — | — | 0.2 | — |
| Xanthan gum *3 | 0.6 | 0.5 | 0.4 | 0.4 | 0.6 | 0.8 | 0.6 | 0.6 |
| 1,3-butylene glycol | 10 | — | 5 | 10 | 4 | 4 | 10 | 10 |
| Dipropylene glycol | — | — | 2 | — | 4 | 4 | — | — |
| Glycerin | — | — | 2 | — | 2 | — | — | — |
| Ascorbyl glucoside | 2 | 2 | — | 0.1 | — | 2 | 2 | 2 |
| Magnesium ascorbyl | — | — | 3 | — | 3 | — | — | — |
| phosphate | | | | | | | | |
| Niacinamide | 3.5 | 1 | 2 | — | 0.5 | — | 3.5 | 3.5 |
| Sodium salicylate | 0.5 | 0.3 | 0.5 | 0.5 | — | 0.5 | 0.5 | 0.5 |
| Disodium phosphate | 0.1 | — | 0.1 | 0.1 | — | — | 0.1 | 0.1 |
| Sodium citrate | — | 1 | 1 | 1 | — | 1 | — | — |
| Sodium hydroxide | 0.24 | 0.21 | — | — | — | 0.21 | 0.24 | 0.24 |
| Polysorbate 20 *4 | 0.3 | — | — | — | 0.5 | 0.2 | 0.3 | 0.3 |
| Perfume | 0.05 | — | — | — | 0.1 | 0.03 | 0.05 | 0.05 |
| Methyl paraben | 0.1 | 0.1 | 0.1 | 0.1 | 0.1 | 0.1 | 0.1 | 0.1 |
| Benzyl alcohol | 0.15 | — | 0.15 | — | 0.15 | 0.15 | 0.15 | 0.15 |
| Phenoxyethanol | — | 0.2 | — | 0.2 | — | — | — | — |
| EDTA-2Na | 0.1 | 0.05 | 0.1 | 0.1 | 0.1 | 0.1 | 0.1 | 0.1 |
| Ascosporogeneous | — | — | — | — | — | — | — | 30 |
| Yeast Fermented | | | | | | | | |
| Filtrate *5 | | | | | | | | |

| | |
|---|---|
| Deionized Water | q.s. to 100% |

Definitions of Components
*1 Titanium dioxide: Kobo GLW75CAP-MP available from Kobo Products Inc.
*2 Resistant starch: Novelose 240 ® available from National Starch and Chemical Company Inc.
*3 Xanthan gum: Keltrol available from Kelco
*4 Polysorbate 20: Tween 20 available from ICI Surfactants
*5 Yeast Ferment Filtrate: SKII Pitera available from Kashiwayama

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

All documents cited in the Detailed Description of the Invention are, in relevant part, incorporated herein by reference; the citation of any document is not to be construed as an admission that it is prior art with respect to the present invention. To the extent that any meaning or definition of a term in this written document conflicts with any meaning or definition of the term in a document incorporated by reference, the meaning or definition assigned to the term in this written document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A treatment article comprising:
a) a first substrate;
b) a second substrate which has a shape to substantially cover the first substrate at-use;
c) a first treatment composition; and
d) a second treatment composition,
wherein the second substrate is a water-insoluble substrate.

2. The article according to claim 1, wherein said first substrate is a water-soluble film.

3. The article according to claim 1, wherein said first substrate is a water-insoluble substrate.

4. The article according to claim 2, wherein said water-soluble film is a multilayer water-soluble film.

5. The article according to claim 1, wherein said first treatment composition and said second treatment composition include at least one skin care active.

6. The article according to claim 5, wherein said first treatment composition differs from said second treatment composition.

7. The article according to claim 6, wherein said first treatment composition further comprises a skin benefit agent which is unstable when contacted with said second treatment composition.

8. The article of claim 5, wherein said skin care active is selected from the group consisting of skin texture improvement agents, anti-acne actives, anti-oxidants, peptides, anti-inflammatory agents, humectants, whitening agents, skin tone changing agents and combinations thereof.

9. A treatment article comprising:
a) a first substrate containing a first treatment composition;
b) a second substrate which has a shape to substantially cover the first substrate at-use; and
c) a second treatment composition,
wherein the second substrate is a water-insoluble substrate and delivery of the first treatment composition to the skin is initiated by contacting the first substrate with the second treatment composition.

10. The article according to claim 9, wherein said first substrate is a water-soluble film.

11. The article according to claim 10, wherein said water-soluble film dissolved within 1-60 minutes when it contacts water.

12. The article according to claim 10, wherein said second treatment composition comprises an aqueous carrier.

13. The article according to claim 9, wherein said first treatment composition contains a skin benefit agent which is unstable when contacted with said second treatment composition.

14. The article according to claim 9, wherein said article is a facial mask.

15. A method of treating the skin comprising the steps of:
a) providing a treatment article comprising a first substrate, a second substrate, a first treatment composition and a second treatment composition wherein the second substrate is a water-insoluble substrate;
b) applying the first substrate with the first treatment composition to a target area of the skin;
c) applying the second substrate with the second treatment composition to the skin in a way to cover the first substrate and other areas of the skin; and
d) allowing the treatment article to remain on the skin for a period of time effective to provide cosmetic treatment.

16. A method of treating the skin comprising the steps of
a) providing a treatment article comprising a first substrate, a second substrate, a first treatment composition and a second treatment composition wherein the second substrate is a water-insoluble substrate;
b) applying the first substrate with the first treatment composition to a part of the second substrate which is to be located on a target area of the skin;
c) applying the second substrate with the second treatment composition to the skin in a way to locate the first substrate on to a target area of the skin, and the other part of the second substrate to cover other areas of the skin; and
d) allowing the treatment article to remain on the skin for a period of time effective to provide cosmetic treatment.

17. A skin care product comprising;
a) a first substrate containing a first treatment composition located in a first compartment; and
b) a second substrate containing a second treatment composition located in a second compartment; the second substrate is water-insoluble and has a shape to substantially cover the first substrate at-use;
wherein the first and the second substrates are not contacted prior to use; and
wherein the first and second compartments are housed in a single package.
